(19) United States
(12) Patent Application Publication
(10) Pub. No.: US 2014/0377188 A1
(43) Pub. Date: Dec. 25, 2014

(54) Oral Composition Indicative Of Proper Tooth Cleaning

(71) Applicant: The Procter & Gamble Company, Cincinnati, OH (US)

(72) Inventors: Ross Strand, Beijing (CN); Yujun Li, Beijing (CN); Yiqun Zhang, Beijing (CN); Xiaoxiao Li, Beijing (CN); Zhong Lei, Beijing (CN)

(21) Appl. No.: 14/312,749

(22) Filed: Jun. 24, 2014

(30) Foreign Application Priority Data
Jun. 24, 2013 (CN) PCT/CN2013/077757

(51) Int. Cl. A61K 8/02; A61Q 11/00

(52) U.S. Cl. CPC A61K 8/0275; A61Q 11/00; A61K 2800/412
USPC 424/10.1

(57) ABSTRACT

The present invention provides an oral care composition, containing at least 0.5% by weight of silica agglomerates, wherein each of the silica agglomerates has: (i) an overall particle size from 200 μm to 2000 μm, and (ii) a crush strength from 0.1N to 5N. The silica particles that make up the silica agglomerate can have an average particle size from 1 μm to 50 μm. The composition can have a viscosity from 10 to 90 BKU. The present invention also provides a method of encouraging proper tooth cleaning by administering the oral care composition of the present invention to a subject's tooth surface. The present invention further provides the use of a silica agglomerate in manufacturing an oral care composition for encouraging proper tooth cleaning

### FIELD OF THE INVENTION

The present invention relates to an oral composition useful for indicating proper tooth cleaning techniques. The present invention also relates to a method of encouraging proper tooth cleaning by applying the oral composition of the present invention onto a subject's tooth surface and brushing such tooth surface. The present invention further relates to the use of a silica agglomerate in manufacturing an oral composition for encouraging proper tooth cleaning

### BACKGROUND OF THE INVENTION

Tooth cleaning is part of oral hygiene and involves the removal of dental plaque from teeth with the intention of preventing cavities (dental caries), gingivitis, periodontal disease, and even some systemic diseases. People routinely clean their own teeth by brushing with a toothbrush and toothpaste. Incorrect or insufficient brushing may be not able to adequately remove the plaque or stop plaque from building up, and even may be harmful to the tooth surface, the gum, and other delicate intraoral membranes.

Electric toothbrushes, which were initially developed and recommended for people with insufficient strength or dexterity problems in their hands, have come into widespread general use. One of the main reasons is that the electric toothbrush provides a timer which the user can follow easily to brush the teeth with proper duration. The effectiveness of electric toothbrushes at reducing plaque formation and gingivitis is believed to be superior to that of conventional manual toothbrushes. However, at least some research finds that most electric toothbrushes are actually no more effective than manual brushes if people using a manual toothbrush brush the teeth in a proper way (Deery C, Heanue M, Deacon S, Robinson P G, Walmsley A D, Worthington H, Shaw W, Glenny A M (March 2004). “The effectiveness of manual versus powered toothbrushes for dental health: a systematic review”. J Dent 32 (3): 197-211). The way brushing is conducted, including the amount of time spent and the force used, can be more important than the choice of brush.

Therefore, there is a need to develop a product which can help the user clean teeth in a proper way. There is also a need to develop a product at a lower cost than electric toothbrushes to provide a proper tooth-cleaning guide with good user compliance. There is also a need to develop a method of helping the user build a habit of proper tooth cleaning, including but not limited to helping the user develop proper tooth cleaning habits, for example, brushing with proper force for appropriate duration.

### SUMMARY OF THE INVENTION

In one aspect, the present invention provides an oral composition comprising at least 0.5% by weight of silica agglomerates, wherein each of the silica agglomerates has: (i) an overall particle size from 200 μm to 2000 μm, and (ii) a crush strength from 0.1N to 5N; wherein silica particles that make up the silica agglomerate has an average particle size from 1 μm to 50 μm; and wherein the composition has a viscosity from 10 to 90 BKU.

In another aspect, the present invention provides a method of encouraging proper tooth cleaning, comprising applying an oral composition of the present invention onto a subject's tooth surface.

In a further aspect, the present invention provides the use of a silica agglomerate in manufacturing an oral composition for encouraging proper tooth cleaning, wherein said silica agglomerate has: (i) an overall particle size from 200 μm to 2000 μm, and (ii) a crush strength from 0.1N to 5N; and wherein silica particles that make up the silica agglomerate has an average particle size from 1 μm to 50 μm.

By formulating specific silica agglomerates into an oral care composition with the appropriate viscosity, the present invention provides a product and method which can indicate and/or encourage proper tooth cleaning. In accordance with some embodiments of the present invention, the present oral care composition can indicate proper force used for tooth cleaning In accordance with some embodiments of the present invention, the present oral composition can encourage a sufficient and complete tooth cleaning. In accordance with some embodiments of the present invention, the present oral composition can provide a proper tooth-cleaning guide with good user compliance.

These and other features, aspects, and advantages of the present invention will become evident to those skilled in the art from the detailed description which follows.

### BRIEF DESCRIPTION OF THE DRAWINGS

While the specification concludes with claims particularly defining and distinctly claiming the invention, it is believed that the invention will be better understood from the following description of the accompanying figures. In the accompanying figures,

FIG. 1A illustrates how the crush strength is calculated from a test curve obtained by a TA AR2000 rheometer;

FIG. 1B illustrates how the crush strength is calculated from a test curve obtained by a TA AR2000 rheometer;

FIG. 2 shows particle size distribution of the oral care compositions according to Example 1 before and after 3-minutes of brushing;

FIG. 3 shows particle size distribution of the oral care compositions according to Example 2 before and after 3-minutes of brushing;

FIG. 4 shows particle size distribution of the oral care compositions according to Example 3 before and after 3-minutes of brushing;

FIG. 5 shows particle size distribution of the oral care compositions according to Example 4 before and after 3-minutes of brushing;

FIG. 6 shows particle size distribution of the oral care compositions according to Example 5 before and after 3-minutes of brushing; and

FIG. 7 shows particle size distribution of the oral care compositions according to Example 6 before and after 3-minutes of brushing.

### DETAILED DESCRIPTION OF THE INVENTION

All percentages and ratios used herein are by weight of total composition, unless otherwise indicated. All percentages, ratios, and levels of ingredients referred to herein are based on the actual amount of the ingredient, and do not include solvents, fillers, or other materials with which the ingredient may be combined as a commercially available product, unless otherwise indicated.

All measurements referred to herein are made at room temperature of about 25° C., unless otherwise specified.

The terms “oral composition” and “oral care composition” are used interchangeably herein, and refer to a product, which in the ordinary course of usage, is not intentionally swallowed for purposes of systemic administration of particular therapeutic agents, but is rather retained in the oral cavity for a time sufficient to contact substantially all of the dental surfaces and/or oral tissues for purposes of oral activity. The oral composition may be in various forms including toothpaste, dentifrice, tooth gel, subgingival gel, mouthrinse, mousse, foam, denture product, mouthspray, lozenge, chewable tablet or chewing gum. The oral composition may also be incorporated onto strips or films for direct application or attachment to oral surfaces.

The term “dentifrice”, as used herein, includes paste, gel, liquid, powder or tablet formulations unless otherwise specified. The dentifrice composition may be a single phase composition or may be a combination of two or more separate dentifrice compositions. The dentifrice composition may be in any desired form, such as deep striped, surface striped, multilayered, having a gel surrounding a paste, or combinations thereof. Each dentifrice composition in a dentifrice comprising two or more separate dentifrice compositions may be contained in a physically separated compartment of a dispenser and dispensed side-by-side.

The term “teeth”, as used herein, refers to natural teeth as well as artificial teeth or dental prosthesis.

The term “particle size”, as used herein, refers to a volume based particle size measured by laser diffraction methods. Laser diffraction measures particle size distributions by measuring the angular variation in intensity of light scattered as a laser beam passes through a dispersed particulate sample. Large particles scatter light at small angles relative to the laser beam and small particles scatter light at large angles. The angular scattering intensity data is then analyzed to calculate the size of the particles responsible for creating the scattering pattern, using the Mie theory of light scattering. The particle size is reported as a volume equivalent sphere diameter. The term “mean particle size” and “average particle size” are used interchangeably herein, and refer to an average value of particle size distribution calculated based on the logarithmic scale. When referring to an agglomerate particulate, the terms “particle size” and “overall particle size” are used interchangeably.

Active and other ingredients useful herein may be categorized or described by their cosmetic and/or therapeutic benefit or their postulated mode of action or function. However, it is to be understood that the active and other ingredients useful herein can, in some instances, provide more than one cosmetic and/or therapeutic benefit or function or operate via more than one mode of action. Therefore, classifications herein are made for the sake of convenience and are not intended to limit an ingredient to the particularly stated application or applications listed.

As used herein, the articles including “a” and “an” when used in a claim, are understood to mean one or more of what is claimed or described.

As used herein, the terms “comprise”, “comprises”, “comprising”, “include”, “includes”, “including”, “contain”, “contains”, and “containing” are meant to be non-limiting, i.e., other steps and other sections which do not affect the end of result can be added. The above terms encompass the terms “consisting of and “consisting essentially of”.

As used herein, the words “preferred”, “preferably” and variants refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

### Silica Agglomerate

According to the present invention, the oral care composition comprises at least 0.5% by weight of silica agglomerates, wherein each of the silica agglomerates has: (i) an overall particle size from 200 μm to 2000 μm, and (ii) a crush strength from 0.1N to 5N; wherein silica particles that make up the silica agglomerate has an average particle size from 1 μm to 50 μm.

The silica agglomerate useful in the present invention is recommended to have an overall particle size from 200 μm to 2000 μm. In a specific embodiment, each of the silica agglomerates has an overall particle size from 250 μm, 300 μm, 350 μm, 400 μm, or 450 μm to 500 μm, 600 μm, 800 μm, 1000 μm or 1500 μm. In an alternative embodiment, each of the silica agglomerates has an overall particle size from 250 μm or 300 μm to 350 μm or 450 μm. In another alternative embodiment, each of the silica agglomerates has an overall particle size from 500 μm or 600 μm to 800 μm or 1000 μm. The particle size of the silica agglomerate should be big enough to make the user feel it during brushing but should not be so big as to make the user uncomfortable or impact the brushing experience negatively.

The silica agglomerate useful in the present invention is recommended to have a crush strength from 0.1N to 5N. In a specific embodiment, each of the silica agglomerates has a crush strength from 0.5N, 0.8N, 1N, 1.3N, 1.5N or 1.8N to 2N, 2.5N, 3N, 3.5N, 4N or 4.5N. In an alternative embodiment, each of the silica agglomerates has a crush strength from 0.8N or 1N to 1.3N or 1.8N. In another alternative embodiment, each of the silica agglomerates has a crush strength from 2N or 2.5N to 3.5N or 4.5N. The crush strength of the silica agglomerate should be in the range of a proper brushing force. Accordingly, when the silica agglomerate breaks down during brushing, the user gets a signal that a proper brushing force is used. The proper brushing force depends on the brushing purpose and/or the user. For example, in the case of a children's formulation, the proper brushing force and therefore the crush strength of the silica agglomerate may be recommended to be from 0.5N to 2N, preferably from 0.8N to 1.5N. It is important to encourage children to clean their teeth properly from the very beginning of their tooth development. In the case of an adult formulation for routine cleaning, the proper brushing force and therefore the crush strength of the silica agglomerate may be recommended to be from 1N to 4N, preferably from 1.5N to 3.3N. Today there are a wide variety of dentifrices designed for many conditions, including cavities, gingivitis, tartar, stained teeth, sensitivity, and so on. In the case of an anti-tartar or anti-calculus formulation, the proper brushing force and therefore the crush strength of the silica agglomerate may be recommended to be from 2N to 5N, preferably from 3N to 4.5N.

According to the present invention, the differences caused by various toothbrushes will be minimized in terms of brushing effect. Whether the toothbrush is a manual brush or an electric brush, or whether the toothbrush has hard bristles or soft bristles, the user can easily find the proper way to handle the toothbrush for brushing the teeth, based on the breakdown of the silica agglomerate under a proper brushing force. For example, a toothbrush with hard bristles tends to hurt the enamel on the tooth surface if the brushing force is too large, while this concern can be eliminated or removed if a reduced brushing force is used.

The silica agglomerate useful in the present invention comprises silica particles having an average particle size from 1 μm to 50 μm. In a specific embodiment, each of the silica agglomerates comprises silica particles having an average particle size from 2 μm, 3 μm, Sum, 8 μm, or 10 μm to 15 μm, 20 μm, 30 μm, 40 μm or 45 μm. In an alternative embodiment, each of the silica agglomerates comprises silica particles having an average particle size from 1.5 μm, 2.5 μm, or 3.5 μm to 5.5 μm, 7.5 μm or 13.5 μm. In another alternative embodiment, each of the silica agglomerates comprises silica particles having an average particle size from 2 μm, 4 μm, or 9 μm to 15 μm, 20 μm or 40 μm. The silica particles used to make up the silica agglomerate should be of a particle size which tends to agglomerate in a relatively fragile way. Preferably, the silica particles used to make up the silica agglomerate should generally be of a particle size which cannot be felt by a user during brushing. Accordingly, when the silica agglomerate breaks down under proper brushing force, the user obtains a signal by feeling a reduction or elimination of the gritty feel caused by the silica agglomerates (but not from the resulting silica particles). Such a reduction or elimination of the gritty feel upon brushing also makes the user have a sense of achievement, and therefore encourages the user to form a good tooth cleaning habit. It is also found that, when the silica agglomerate is made from silica particles having a specific average particle size, the silica agglomerate can have a good stability during the product's shelf life, while being ready to break down during use, under a proper brushing force, into imperceptible silica particles evenly.

The silica particles used to make up the silica agglomerate can have any suitable crush strength. In a specific embodiment, the silica particles making up the agglomerate have a crush strength that exceeds the crush strength of the agglomerates. The crush strength of the silica particles making up the agglomerate may be greater than 7.5N, 10N, 20N or 50N.

The silica used to make up the silica agglomerate can be selected from the group consisting of fused silica, fumed silica, pyrogenic silica, colloidal silica, precipitated silica, hydrophobic silica, silica gel, aerogel silica, and mixtures thereof. In a preferred embodiment, the silica agglomerate comprises silica particles selected from the group consisting of fumed silica, pyrogenic silica, precipitated silica, silica gel, and mixtures thereof. Silica has been widely used in oral care compositions, including thickening silica and abrasive silica. The silica agglomerate can comprise either of thickening silica and abrasive silica, or a mixture thereof.

Some examples of the silica which can be used to make up the silica agglomerate are those available from Ineos Silicas, Warrington, England, marketed under Sorbosil®; from Rhodia Silica Systems, Lyon, France, marketed under Tixosil® and Oralsil®; from Degussa AG, Germany, marketed under Aerosil® and Sident®; from W. R. Grace & Company, Davison Chemical Division, Columbia, Md., United States, marketed under Syloid®; from J. M. Huber Corporation, Edison, N.J., United States, marketed under Zeodent®; from Cabot Corporation, Bellerica, Mass., United States, marketed under Cab-O-Sil®; and from Millennium Inorganic Chemicals Corporation, Baltimore, Md., United States, marketed under Silcron®.

The silica agglomerate can be made from commercially available silica particles, for example, those selected from Zeodent® 153, Zeodent® 163, Zeodent® 165, Zeodent® 113, Zeodent® 124, Zeodent® 103, Zeodent® 119, Zeodent® 109, Tixosil® 43, Tixosil® 331, Tixosil® 63, Tixosil® 73, Tixolex® 28 or any mixtures thereof. The silica particles may be or may be not structurally modified. Suitable industrial processes for making silica agglomerates from the smaller silica particles include, but are not limited to, wetting and subsequent drying, pressure compaction, and any other possible process. In a specific embodiment, the silica agglomerate is made from silica particles only. In an alternative embodiment, the silica agglomerate is comprised of silica particles and one or more binders. In another alternative embodiment, the silica agglomerate further comprises one or more ingredients selected from the group consisting of an antibacterial agent, a colorant, a flavoring component, and mixtures thereof. Alternatively, the silica agglomerate can be selected from commercially available ones, for example, Tixosil® G and Zeodent® 9175.

In a specific embodiment, the oral care composition comprises 1%, 2%, 3%, 4% or 5% to 6%, 9%, 15%, 20% or 30% by weight of silica agglomerates. In an alternative embodiment, the oral care composition comprises 1.5%, or 2.5% to 3.5% or 4.5% by weight of silica agglomerates. In another alternative embodiment, the oral care composition comprises 5.5%, 8%, or 12% to 18%, 23%, or 28% by weight of silica agglomerates.

In a specific embodiment, the oral care composition comprises 2% to 8% by weight of silica agglomerates, wherein each of the silica agglomerates has: (i) a particle size from 300 μm to 600 μm, and (ii) a crush strength from 1N to 3N; and wherein each of the silica agglomerates comprises silica particles having an average particle size from 5 μm to 20 μm.

In another specific embodiment, the oral care composition comprises 1% to 5% by weight of silica agglomerates, wherein each of the silica agglomerates has: (i) a particle size from 100 μm to 400 μm, and (ii) a crush strength from 0.5N to 2.5N; and wherein each of the silica agglomerates comprises silica particles having an average particle size from 5 μm to 20 μm.

### Viscosity-Control System

It has been surprisingly found that, when the oral care composition comprising the silica agglomerate has a certain viscosity, the silica agglomerates readily spread evenly on the tooth surface as the toothbrush moves around, so as to make sure that every location on the tooth surface is reached and properly cleaned by the toothbrush. It is not easy to control brushing force, especially control with an even and constant brushing force during the whole brushing action. Therefore, the even distribution of the silica agglomerates on teeth surface is important in the sense that the proper brushing force is evenly exerted on the teeth surface. This indicates and encourages efficient and thorough cleaning. Surprisingly, when the oral care composition comprising the silica agglomerate has a certain viscosity, the silica agglomerate has a good agglomerating stability during the manufacturing process of the oral care composition, while being ready to break down into small particles under a proper brushing force.

According to the present invention, the oral care composition has a viscosity from 10 to 90 BKU. In a specific embodiment, the oral care composition has a viscosity from 15, 20, 25 or 30 to 40, 50, 60, or 70 BKU. In an alternative embodiment, the oral care composition has a viscosity from 12 or 18 to 28 or 33 BKU. In another alternative embodiment, the oral care composition has a viscosity from 35 or 45 to 55 or 65 BKU. As used herein, “BKU” is the unit of Brookfield viscosity. Brookfield viscosity is determined on a Brookfield RVT 1/2 Heliopath Viscometer using an E spindle set at 2.5 rpm rotational speed spindle at room temperature.

A thickening agent is generally used in an oral care composition to control or modify the viscosity of the composition. The thickening agent useful in the present invention is selected from the group consisting of polysaccharides or polysaccharide derivatives (e.g., methylcellulose, ethylcellulose, carboxymethylcellulose, hydroxyethylcellulose, hydroxypropylcellulose, hydroxy-propylmethylcellulose, and other cellulose derivatives), carbomers (e.g., crosslinked polyacrylic acid copolymer or homopolymer and copolymers of acrylic acid cross linked with a polyalkenyl polyether), natural and synthetic gums (e.g., carrageenan, xanthan gum, karaya gum, guar gum, gelatin, algin, sodium alginate, tragacanth gum, chitosan, acacia gum, etc.), acrylamide polymers, acrylic acid polymers, vinyl polymers (e.g., polyvinyl alcohol, polyvinylpyrrolidone, etc.), polyamines, polyquarternary compounds, ethylene oxide polymers, and mixtures thereof. Some inorganic thickening agents, including but not limited to mineral oil, petrolatum, clays and organomodified clays, silica and the like, can also be used in the present invention.

The thickening agent is present in an amount from 0.01% to 20% by weight of the oral care composition. In a specific embodiment, the thickening agent is present in an amount from 0.1%, 0.5%, 1%, or 2% to 3%, 5%, 8% or 15% by weight of the oral care composition. In another specific embodiment, the thickening agent is present in an amount from 0.2%, 0.3%, 0.5%, or 0.8% to 1%, 2%, 3%, or 5% by weight of the oral care composition.

The thickening agent may be used with or without a carrier. Examples of the suitable carrier include but not limited to glycerol, polyethylene glycol (e.g., PEG-400), or mixtures thereof. When a carrier is used, preferably up to 5%, more preferably from 0.1% to 1% of thickening agent, is combined with preferably from 95% to 99.9%, more preferably from 99% to 99.9% of carrier, based on the total weight of the thickening agent/carrier combination. Furthermore, when the thickening agent is a hydrated silica and it is used with a carrier, preferably from 5% to 10% of thickening agent is combined with preferably from 90% to 95% of carrier, based on the total weight of the thickening agent/carrier combination.

In a specific embodiment, the present oral care composition comprises a thickening agent selected from carbomers, e. g. the class of homopolymers of acrylic acid crosslinked with an alkyl ether of pentaerythritol or an alkyl ether of sucrose. Carbomers are commercially available from B. F. Goodrich as the Carbopol® series, including Carbopol® 934, Carbopol® 940, Carbopol® 941, Carbopol® 956, and mixtures thereof. Homopolymers of polyacrylic acid are described, for example, in U.S. Pat. No. 2,798,053. Such polymers are homopolymers of unsaturated, polymerizable carboxylic monomers such as acrylic acid, methacrylic acid, maleic acid, itaconic acid, maleic anhydride, and the like.

In another specific embodiment, the present oral care composition comprises a thickening agent selected from the group consisting of methylcellulose, ethylcellulose, hydroxyethylcellulose, carboxymethylcellulose, carrageenan, xanthan gum, guar gum, tragacanth gum, alginate, acacia gum, gelatin, and mixtures thereof, preferably in an amount from 0.2%, 0.3%, 0.6%, or 0.8% to 1.1%, 1.4%, 1.8%, or 2.5% by weight of the oral care composition. In a further preferred embodiment, the present oral care composition comprises a thickening agent selected from the group consisting of hydroxyethylcellulose, carrageenan, sodium carboxymethylcellulose, and mixtures thereof, preferably in an amount from 0.2%, 0.3%, or 0.4% to 0.6%, 0.8% or 1.5% by weight of the oral care composition.

### Optional Components

The oral composition of the present invention can contain a variety of optional conventional oral composition components. Such optional components include, but not limited to, humectant, surfactant, an antibacterial agent, fluoride ion source, and some other conventional components useful in the oral composition. The silica agglomerate used in the present invention can further comprise one or more ingredients selected from the group consisting of an antibacterial agent, a colorant, a flavorant, and mixtures thereof, to provide further benefit in terms of controlled release and/or signaling.

Humectants such as polyethylene glycols can also been used in dentifrice compositions to modify viscosity and to provide a smooth feel to dentifrice compositions. Polyethylene glycols are available in a large range of average molecular weights and have different properties depending upon their average molecular weights. The humectant serves to keep the oral composition, especially a toothpaste composition, from hardening upon exposure to air and give a moist feel to the mouth. Certain humectants can also impart a desirable sweet flavor to oral compositions such as mouth-wash and toothpaste. Suitable humectants for use in the present invention include edible polyhydric alcohols such as glycerin, sorbitol, xylitol, butylene glycol, polyethylene glycol, propylene glycol, and mixtures thereof. The humectant is optionally present in a total amount of 1% to 70%, for example 1% to 50%, 2% to 25%, or 5% to 15% by weight of the composition.

Surfactants are useful, for example, to compatibilize other components of the composition and thereby provide enhanced stability, to help in cleaning the dental surface through detergency, and to provide foam upon agitation, e.g., during brushing with a dentifrice composition of the invention. Any orally acceptable surfactant, most of which are anionic, nonionic or amphoteric, can be used. Suitable anionic surfactants include without limitation water-soluble salts of C8-20 alkyl sulfates, sulfonated monoglycerides of C8-20 fatty acids, sarcosinates, taurates and the like. Illustrative examples of these and other classes include sodium lauryl sulfate, sodium coconut monoglyceride sulfonate, sodium lauryl sarcosinate, sodium lauryl isoethionate, sodium laureth carboxylate and sodium dodecyl benzenesulfonate. Suitable nonionic surfactants include without limitation poloxamers, polyoxyethylene sorbitan esters, fatty alcohol ethoxylates, alkylphenol ethoxylates, tertiary amine oxides, tertiary phosphine oxides, dialkyl sulfoxides and the like. Suitable amphoteric surfactants include without limitation derivatives of C8-20 aliphatic secondary and tertiary amines having an anionic group such as carboxylate, sulfate, sulfonate, phosphate or phosphonate. A suitable example is cocoamidopropyl betaine. The surfactant is optionally present in a total amount of 0.01% to 10%, for example 0.05% to 5% or 0.1% to 2% by weight of the composition.

The present oral care composition can incorporate a soluble fluoride source, also called a fluoride ion source, capable of providing free fluoride ions. Preferred fluoride ion sources are selected from the group consisting of sodium fluoride, stannous fluoride, sodium monofluorophosphate, amine fluoride, and combinations thereof. Sodium fluoride is the most preferred soluble fluoride ion source. Norris et al., U.S. Pat. No. 2,946,725, issued Jul. 26, 1960, and Widder et al., U.S. Pat. No. 3,678,154 issued Jul. 18, 1972, disclose such fluoride ion sources as well as others. In specific embodiments, the present oral composition contains a fluoride ion source capable of providing from 50 ppm to 3500 ppm, preferably from 500 ppm, 1000 ppm, or 1500 ppm to 2000 ppm, 2500 ppm, or 3000 ppm of free fluoride ions.

Antibacterial agents useful in the present invention includes but not limited to water insoluble non-cationic antibacterial agents and water soluble antibacterial agents such as quaternary ammonium salts and bis-biquanide salts are suitable for inclusion among others. Triclosan monophosphate is an additional water soluble antibacterial agent. In some preferred embodiments, the antibacterial agent is selected from the group consisting of cetylpyridinium halide, domiphen halide, stannous ion source, zinc ion source, copper ion source, and combinations thereof. These antibacterial agents may be present at levels of from 0.01%, 0.05%, 0.1%, or 0.2% to 0.5%, 1.0%, 1.2% or 1.5% by weight.

Colorants herein include pigments, dyes, lakes and agents imparting a particular luster or reflectivity such as pearling agents. A colorant can serve a number of functions, including for example to provide a white or light-colored coating on a dental surface, to act as an indicator of locations on a dental surface that have been effectively contacted by the composition, and/or to modify appearance, in particular color and/or opacity, of the composition to enhance attractiveness to the user. Any orally acceptable colorant can be used, including but not limited to talc, mica, magnesium carbonate, calcium carbonate, magnesium silicate, magnesium aluminum silicate, silica, titanium dioxide, zinc oxide, red, yellow, brown and black iron oxides, ferric ammonium ferrocyanide, manganese violet, ultramarine, titaniated mica, bismuth oxychloride and the like.

Flavorants are useful for example to enhance taste of the composition. Any orally acceptable natural or synthetic flavorant can be used, including but not limited to vanillin, sage, marjoram, parsley oil, spearmint oil, cinnamon oil, oil of wintergreen (methylsalicylate), peppermint oil, clove oil, bay oil, anise oil, eucalyptus oil, citrus oils, fruit oils and essences including those derived from lemon, orange, lime, grapefruit, apricot, banana, grape, apple, strawberry, cherry, pineapple, etc., bean- and nut-derived flavors such as coffee, cocoa, cola, peanut, almond, etc., adsorbed and encapsulated flavorants and the like. Also encompassed within flavorants herein are ingredients that provide fragrance and/or other sensory effect in the mouth, including cooling or warming effects. Such ingredients illustratively include menthol, menthyl acetate, menthyl lactate, camphor, eucalyptus oil, eucalyptol, anethole, eugenol, cassia, oxanone, α-irisone, propenyl guaiethol, thymol, linalool, benzaldehyde, cinnamaldehyde, N-ethyl-p-menthan-3-carboxamine, N,2,3-trimethyl-2-isopropylbutanamide, 3-(1-menthoxy)-propane-1,2-diol, cinnamaldehyde glycerol acetal (CGA), menthone glycerol acetal (MGA) and the like.

In a specific embodiment, the oral care composition is in a form selected from the group consisting of toothpaste, tooth powder, tooth gel, and mixtures thereof.

### The Method, Use and Kit

The present invention also relates to a method of encouraging proper tooth cleaning, comprising the step of administering to a subject's tooth surface an oral care composition of the present invention.

In a specific embodiment, the method comprises the step of brushing the subject's tooth surface with the oral care composition of the present invention. The benefits of the present oral composition may increase over time when the composition is used repeatedly.

The subject may be any human or animal whose tooth surface and oral cavity need to be treated with the present oral composition. “Animal” is meant to include household pets or other domestic animals, or animals kept in captivity.

The present invention further relates to the use of a silica agglomerate in manufacturing an oral care composition for encouraging proper tooth cleaning, wherein said silica agglomerate has: (i) an overall particle size from 200 μm to 2000 μm, and (ii) a crush strength from 0.1N to 5N; and wherein silica particles that make up the silica agglomerate has an average particle size from 1 μm to 50 μm.

The present invention further relates to a kit comprising the composition of the present invention and comprising instructions for use. The instructions, in a specific embodiment, instruct the user to continue brushing teeth with the composition until the “gritty feeling” is reduced significantly or eliminated.

### EXAMPLES

The examples herein are meant to exemplify the present invention but are not used to limit or otherwise define the scope of the present invention.

### Silica Agglomerates

Three kinds of silica agglomerate are prepared from Zeodent® 165, Zeodent® 109 and Zeodent® 119 (all commercially available from J. M. Huber Corporation, Edison, N.J., United States), respectively. Taking Zeodent® 165 as an example, the silica agglomerates of Zeodent® 165 are prepared as follows: weigh a certain amount of Zeodent® 165, add water to the weighed Zeodent® 165 with a water to silica ratio of 1.5 to 1, granulate the blend in a pan granulator to provide wet agglomerates, dry the wet agglomerates for 4 hours in an oven at 1200° C., and screen the dried agglomerates to obtain those agglomerates which can pass through a sievepore of 600 μm but cannot pass through a sievepore of 300 μm.

The crush strength of the silica agglomerates is tested on a TA AR2000 rheometer (available from TA Instruments, New Castle, United States). Geometry is 40 mm steel parallel plate. A single silica agglomerate is randomly picked up and put on the Peltier plate. For each of the three kinds of silica agglomerates, five single silica agglomerates are tested to get an average result. Squeeze/pull off test is conducted with the following test settings:
Gap speed (micro m/s): 10.0 (Compression)
Distance (micro m): 500
Sample points: 250
Step termination: When normal force greater than 40.00 N

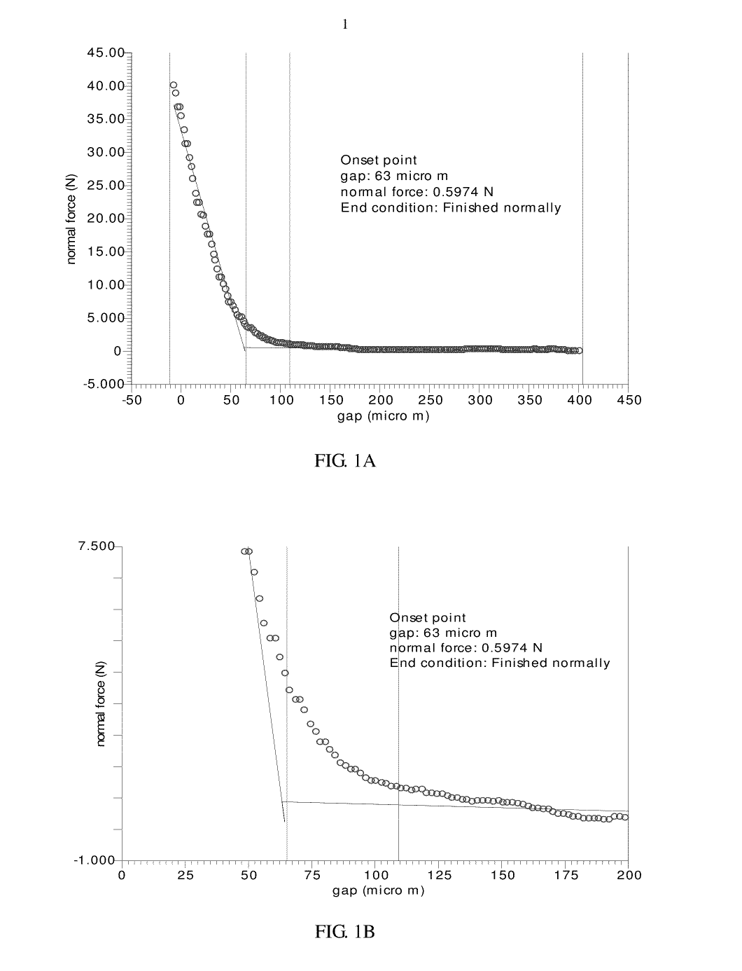

FIGS. 1a to 1b show how the crush strength is calculated: draw fitted lines for the segments in the test curve before and after crush, respectively, identify the point at which each of the fitted lines starts to separate from the test curve as a separating point, define the segment between the separating points of the two fitted lines as an inflection area, average the normal forces of all the points within the inflection area as the crush strength. Table 1 shows the crush strength for the three kinds of silica agglomerates.

TABLE 1

| | | |
|---|---|---|
| Silica | Silica | Silica |
| agglomerate of | agglomerate of | agglomerate of |
| Zeodent 165 | Zeodent 109 | Zeodent 119 |

| | | | |
|---|---|---|---|
| Crush strength/N | 1.73 | 1.33 | 1.68 |

### Oral Care Compositions

Six examples of the oral care compositions are shown in Table 2. All ingredient amounts are described in weight percentages (wt %) unless otherwise indicated. The oral care compositions are prepared as follows: add water, humectants, part of flavor, colorant, buffer and active to a main mixing tank of 35° C., mixing well and ensuring all the ingredients have dissolved or been well dispersed; add thickening agents and sweetener into the main mixing tank, mixing and homogenizing until well dispersed and homogeneous; add abrasive silica and silica agglomerates, mixing and homogenizing until well dispersed and homogeneous; deaerate; add surfactant solution, rest part of flavor to the main mixing tank, mixing and homogenizing until homogeneous; deaerate; pump out and cool the batch to less than 40° C.

TABLE 2

| INGREDIENTS | Ex. 1 | Ex. 2 | Ex. 3 | Ex. 4 | Ex. 5 | Ex. 6 |
|---|---|---|---|---|---|---|
| Sorbitol | 14.18 | 28.35 | 28.35 | 28.35 | 28.35 | 24.65 |
| Sodium Carboxymethyl Cellulose | 0.50 | 0.50 | 1.00 | 1.00 | 0.50 | 0.87 |
| Hydroxyethylcellulose | 0.15 | 0.15 | 0.30 | 0.30 | 0.15 | 0.26 |
| Carrageenan | 0.25 | 0.25 | 0.50 | 0.50 | 0.25 | 0.43 |
| Saccharin Sodium | 0.15 | 0.30 | 0.30 | 0.30 | 0.30 | 0.26 |
| Sodium Fluoride | 0.24 | 0.24 | 0.24 | 0.24 | 0.24 | 0.24 |
| Sodium Citrate Dihydrate | 0.14 | 0.27 | 0.27 | 0.27 | 0.27 | 0.23 |
| Zinc Citrate Dihydrate | 0.40 | 0.79 | 0.79 | 0.79 | 0.79 | 0.69 |
| Polyethylene Glycol 300 | 40.00 | — | — | — | — | 13.04 |
| Abrasive Silica | 4.00 | 20.00 | 17.00 | 17.00 | 20.00 | — |
| Sodium Lauryl Sulfate solution | 1.05 | 2.10 | 2.10 | 2.10 | 2.10 | 1.82 |
| Flavor | 0.60 | 1.20 | 1.20 | 1.20 | 1.20 | 1.05 |
| Colorant | 0.20 | 0.40 | 0.40 | 0.40 | 0.40 | 0.35 |
| Silica agglomerate of Zeodent 165 | — | 2.00 | 2.00 | 5.00 | 8.00 | 14.78 |
| Silica agglomerate of Zeodent 109 | 3.50 | — | — | — | — | — |
| Silica agglomerate of Zeodent 119 | 7.50 | — | — | — | — | 6.96 |
| Treated Water | 27.14 | 43.45 | 45.55 | 42.55 | 37.45 | 34.37 |
| Total | 100 | 100 | 100 | 100 | 100 | 100 |

Viscosity is measured by a Brookfield Digital Viscometer with T-E spindle, Model 1/2 RVT (½ spring strength), with a Brookfield “Helipath” stand. The T-E spindle is a conventional “E-series” Tshaped spindle. The viscometer is placed on the Helipath stand and leveled via spirit levels. The T-E spindle is attached, and the viscometer is set to 2.5 RPM while it is running The viscosity is measured after 10 minutes and the temperature is constant, at 25° C. Table 3 shows the viscosity of each oral care composition.

TABLE 3

| | Ex. 1 | Ex. 2 | Ex. 3 | Ex. 4 | Ex. 5 | Ex. 6 |
|---|---|---|---|---|---|---|
| Viscosity/BKU | 15 | 27 | 49 | 69.8 | 85.6 | 107.6 |

### Brushing Test

Brushing test is performed to demonstrate the efficacy of the present oral care composition. Particle size change is measured before and after brushing. Oral-B ProfessionalCare SmartSeries 5000 with SmartGuide Electric Toothbrush is used and equipped with an Oral-B Precision Clean Electric Toothbrush Head. A watch glass with diameter 70 mm is used as a surface on which the brushing is preformed.

The brushing procedure is as follows: immerse toothbrush in deionized water (DI water) at room temperature for at least 1 min prior to use, wet the watch glass with DI water and pour off excess water, weigh 0.65 g±0.02 g toothpaste onto the bristles of the toothbrush with the balance, and brush the toothpaste on the watch glass for 3 mins with a press force of 2.5N.

### Particle Size Analysis

The particle size of the particulates in the oral care composition is measured using a laser diffraction particle sizing instrument (Mastersizer 2000 from Malvern Instruments). The laser diffraction technique works by measuring the light scattered from particulates as they pass through a laser beam. Particulates scatter light at an angle that is directly related to their size. The Mastersizer 2000 uses the light scattering pattern associated with a sample to calculate particle size distributions. The instrument follows the recommendations of ISO 13320-1-1999.

A dispersion containing the oral care composition is prepared for the measurement. The instrument is connected to a jacketed beaker containing the dispersion to be measured. The dispersion is re-circulated between the beaker and the sampling cell of the particle sizing instrument where the particle size is measured. In order to reduce the sampling variation, the whole brushing glass plate is put into measurement beaker and stirred until all of samples are solved into DI water. Particle size distribution, D90 and mean particle size (mean PS) are obtained for each sample. D90 means the particle size of no more than 90% of the total amount of particles. For example, a D90 of 50 nm means that no more than 90% of the total amount of particles may have a particle size of 50 nm or less. Mean PS means an average particle size of all the particles. For each measurement, four records are created wherein three of them relate to the measurement of the sample, and the fourth is an average result.

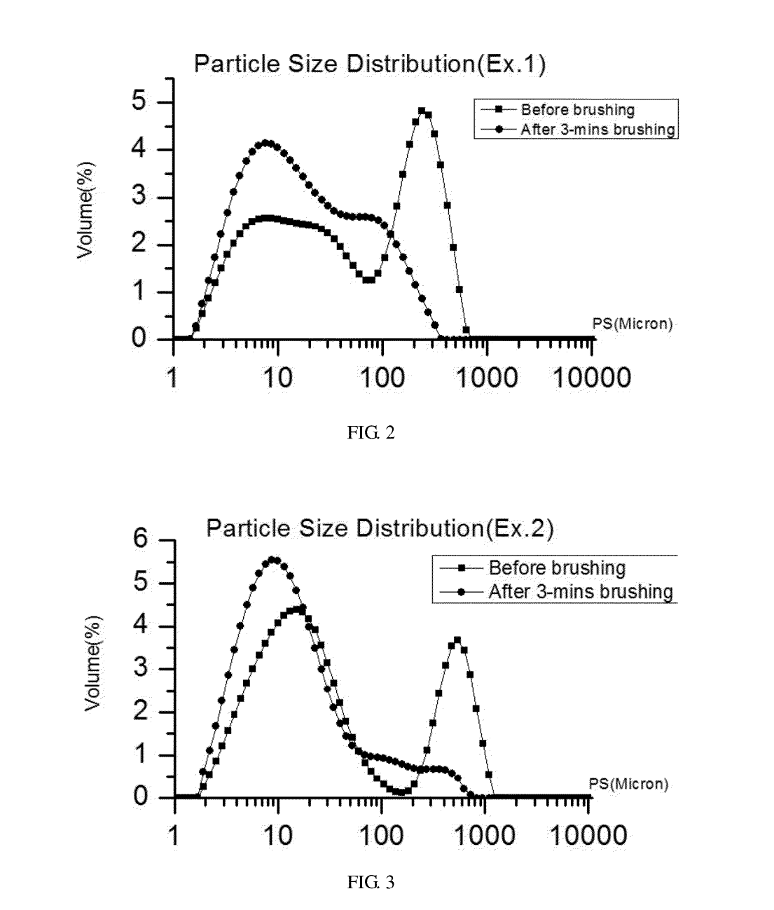

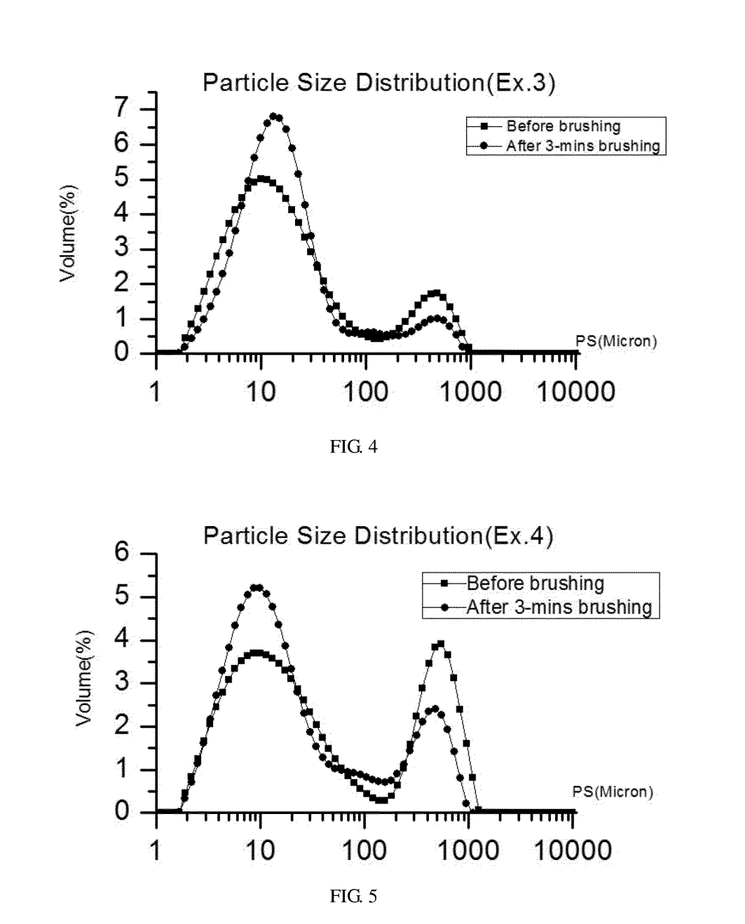

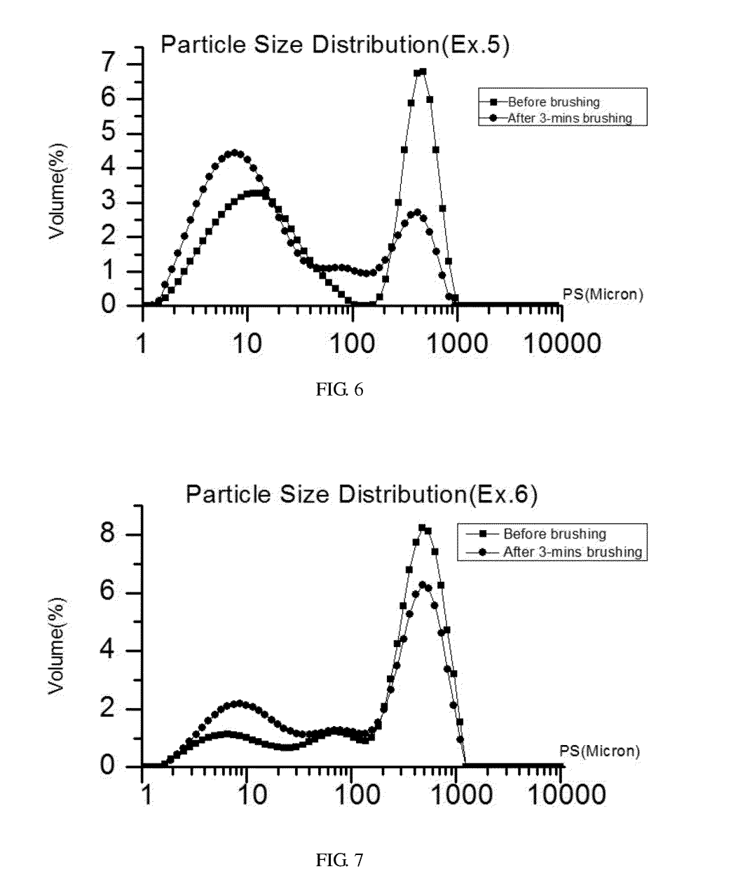

FIGS. 2 to 7 show the particle size distribution for each oral care composition before and after brushing. Table 4 shows a summary of D90 and Mean PS for each oral care composition before and after brushing.

TABLE 4

| | | Ex. 1 | Ex. 2 | Ex. 3 | Ex. 4 | Ex. 5 | Ex. 6 |
|---|---|---|---|---|---|---|---|
| Before brushing/μm | D90 | 313.032 | 553.068 | 309.378 | 583.939 | 612.9157 | 714.951 |
| | Mean PS | 114.746 | 151.074 | 75.751 | 172.293 | 229.896 | 350.003 |
| After 3-min brushing/μm | D90 | 106.624 | 85.759 | 105.412 | 394.518 | 411.8417 | 649.67 |
| | Mean PS | 37.305 | 37.111 | 49.873 | 97.674 | 109.664 | 268.145 |

In FIGS. 2 to 6, we can see two distinct peaks for each before-brushing sample. The peak in smaller particle size range indicates the presence of abrasive silica with an average particle size of about 13 nm. The peak in larger particle size range indicates the presence of silica agglomerates. FIG. 7 shows the particle size distribution of the particulates in the oral care composition of Ex. 6. No abrasive silica with an average particle size of about 13 nm is intentionally added into Ex. 6, but there are two minor peaks in the smaller particle size range, which may indicate some breakdown of the silica agglomerates during the preparation process of the composition.

Comparing the particle size distribution curve of each sample before and after brushing, it can be seen that the peak in smaller particle size range becomes higher and/or broader after brushing, while the peak in larger particle size range becomes lower or narrower after brushing. This indicates the breakdown of silica agglomerate into small particles during brushing.

A lower viscosity of the oral care composition is recommended. As shown in FIGS. 2 and 3, when the viscosity of the oral care composition is as low as 15 BKU and 27 BKU, respectively, the silica agglomerates significantly decrease after brushing. As the viscosity of the oral care composition increases, the change of the peak in larger particle size range becomes less significant (see, FIG. 7, especially compared with FIG. 2). As shown in Table 4, both D90 and mean PS of the oral care compositions show a decrease before and after brushing. The lower the viscosity of the composition is, the more significant the decrease is.

Unless otherwise indicated, all percentages, ratios, and proportions are calculated based on weight of the total composition. All temperatures are in degrees Celsius (° C.) unless otherwise indicated. All measurements made are at 25° C., unless otherwise designated. All component or composition levels are in reference to the active level of that component or composition, and are exclusive of impurities, for example, residual solvents or by-products, which may be present in commercially available sources.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An oral care composition, comprising at least 0.5% by weight of silica agglomerates, wherein each of the silica agglomerates has: (i) an overall particle size from 200 μm to 2000 μm, and (ii) a crush strength from 0.1N to 5N; wherein silica particles that make up the silica agglomerates have an average particle size from 1 μm to 50 μm; and wherein the composition has a viscosity from 10 to 90 BKU.

2. The oral care composition according to claim 1, wherein said oral care composition has a viscosity from 15 to 70 BKU.

3. The oral care composition according to claim 1, wherein said silica particles that make up the silica agglomerates are selected from the group consisting of fumed silica, pyrogenic silica, precipitated silica, silica gel, and mixtures thereof.

4. The oral care composition according to claim 1, wherein said oral care composition comprises 1% to 30% by weight of said silica agglomerates.

5. The oral care composition according to claim 1, wherein said silica particles that make up the silica agglomerates have an average particle size from 2 μm to 45 μm.

6. The oral care composition according to claim 1, wherein each of said silica agglomerates has an overall particle size from 250 μm to 1500 μm.

7. The oral care composition according to claim 1, wherein each of said silica agglomerates has a crush strength from 0.5N to 4N.

8. The oral care composition according to claim 1, wherein each of said silica agglomerates has: (i) an overall particle size from 300 μm to 600 μm, and (ii) a crush strength from 1N to 3N.

9. The oral care composition according to claim 1, wherein said oral care composition further comprises a thickening agent selected from the group consisting of polysaccharides and derivatives thereof, carbomers, natural and synthetic gums, acrylamide polymers, acrylic acid polymers, vinyl polymers, polyamines, ethylene oxide polymers, mineral oils, petrolatums, clays and organomodified clays, and mixtures thereof.

10. The oral care composition according to claim 9, wherein the thickening agent is selected from the group consisting of methylcellulose, ethylcellulose, hydroxyethylcellulose, carboxymethylcellulose, carrageenan, xanthan gum, guar gum, tragacanth gum, alginate, acacia gum, gelatin, and mixtures thereof.

11. The oral care composition according to claim 9, wherein said thickening agent is present in an amount from 0.2% to 5% by weight of said oral care composition.

12. The oral care composition according to claim 11, wherein the thickening agent is present in said oral care composition in an amount from 0.4% to 1.5% by weight, and wherein the thickening agent is selected from the group consisting of hydroxyethylcellulose, carrageenan, sodium carboxymethylcellulose, and mixtures thereof.

13. The oral care composition according to claim 1, wherein said oral care composition comprises a surfactant selected from the group consisting of glycerin, sorbitol, xylitol, butylene glycol, polyethylene glycol, propylene glycol, and mixtures thereof.

14. The oral care composition according to claim 1, wherein each of said silica agglomerates further comprises one or more ingredients selected from the group consisting of an antibacterial agent, a colorant, a flavorant, and mixtures thereof.

15. The oral care composition according to claim 1, wherein said oral care composition is in a form selected from the group consisting of toothpaste, tooth powder, tooth gel, and mixtures thereof.

16. A method of encouraging proper tooth cleaning, comprising the steps of applying the oral care composition according to claim 1 onto a subject's tooth surface and brushing said tooth surface with said oral care composition.

17. Use of a silica agglomerate in manufacturing an oral care composition, wherein said silica agglomerate has: (i) a particle size from 200 μm to 2000 μm, and (ii) a crush strength from 0.1N to 5N; and wherein silica particles that make up the silica agglomerates have an average particle size from 1 μm to 50 μm.

18. The use according to claim 17, wherein said oral care composition has a viscosity from 10 to 90 BKU.

19. The use according to claim 17, wherein said oral care composition is in a form selected from the group consisting of toothpaste, tooth powder, tooth gel, and mixtures thereof.
